(19) United States
(12) Patent Application Publication
(10) Pub. No.: US 2016/0243020 A1
(43) Pub. Date: Aug. 25, 2016

(54) COMPOSITION

(71) Applicant: CONOPCO, INC., D/B/A UNILEVER, Englewood Cliffs, NJ (US)

(72) Inventors: Nicholas BALLARD, Donostia-San Sebastian (ES); Stefan Antonius Franciscus BON, Birmingham (GB); Abid IFTIKHAR, Liverpool (GB); Ezat KHOSHDEL, Neston, Cheshire (GB)

(73) Assignee: Conopco, Inc., d/b/a UNILEVER, Englewood Cliffs, NJ (US)

(21) Appl. No.: 15/029,711

(22) PCT Filed: Oct. 22, 2014
(86) PCT No.: PCT/EP2014/072608

§ 371 Date: Apr. 15, 2016

(30) Foreign Application Priority Data
Oct. 22, 2013 (EP) 13189643.3

(51) Int. Cl. A61K 8/81; A61K 8/37; A61K 8/73; A61Q 5/02; A61K 8/04

(52) U.S. Cl. CPC A61K 8/8152; A61Q 5/02; A61K 8/046; A61K 8/8117; A61K 8/731; A61K 8/37; A61K 2800/412; A61K 2800/41

(57) ABSTRACT

Dry shampoo composition comprising a base and propellant, wherein the base comprises sebum absorber particles, said particles comprising acrylate cross-polymer, polydivinyl benzene polymers or or hydrohobically modified polysaccharide derivatives, wherein the particles are insoluble in water.

The present invention relates to a dry shampoo.

Dry shampoos are compositions for cleansing the hair without washing with water. The product provides a suitable product to provide a clean feel and look on days when perhaps there is no opportunity, either through lack of time or lack of available water, to wash the hair.

Database GNPD [Online] Mintel November 2011 (2011-11) “Instant Dry Shampoo” XP002683579 Database accession no. 1669958 discloses a dry shampoo comprising aluminium starch octenylsuccinate, silica and glycerine. The composition also comprises butylenes glycol.

Database GNPD [Online] Mintel February 2011 (2012-02) “Instant Dry Shampoo” XP002683580 Database accession no. 1727707 discloses a dry shampoo comprising aluminium starch octenylsuccinate, silica and glycerine. The composition also comprises butylenes glycol.

Despite the prior art there remains a need for improved dry shampoos. In particular there is a need for dry shampoos to provide sebum removal, i.e. cleansing of the hair and also to provide volume to the hair.

We have surprisingly found that an improved dry shampoo can be formulated with synthetic particles. The particles are applied to the hair in the usual manner and removed by applying a comb or brush through the hair. The consumer is provided with a simple yet effective means for cleaning the hair on a non-wash day.

Accordingly, and in a first aspect, the present invention provides a dry shampoo composition comprising a base and propellant, wherein the base comprises sebum absorber particles, said particles comprising acrylate cross-polymer, polydivinyl benzene polymers or or hydrohobically modified polysaccharide derivatives, and wherein the particles are insoluble in water.

Preferably, the composition comprises substantially no alcohol. More preferably the composition comprises substantially no ethanol. More preferably, the composition comprises from 0 to 2% wt., most preferably 0 to 1% wt. of the composition alcohol. More preferably, the composition comprises from 0 to 2% wt., more preferably from 0 to 1% wt. of the composition ethanol.

Preferably, the base comprises from 5 to 20% wt. of the composition.

Preferably, the base comprises from 2 to 75% wt of the base sebum absorber.

The particles are preferably crosslinked, preferably at a level of 20-90%, more preferably 50-90%, most preferably 80 -90%.

Preferably, the particles comprise styrene/polydivinylbenzene copolymer.

More preferably, the particles comprise hydrophobic acrylate, for example, methyl acrylate, ethyl acrylate, butyl acrylate, ethylhexyl acrylate, octyl acrylate, decyl acrylate, lauryl acrylate, hexadecyl acrylate and octadecyl acrylate.

The particles may comprise a hydrohobically modified polysaccharide derivative. Preferred hydrohobically modified polysaccharide derivatives are hydrophobically modified cellulose derivatives. A preferred derivative is triacetate. An even more preferred cellulose derivative is cellulose acetate Butyrate, available from, from example, Eastman.

Preferably, the sebum absorbers have an average particle size d(0.5) as measured using a Malvern (DLS) Mastersizer 2000 from 2 to 120 microns, more preferably from 5 to 90 microns.

Preferred particles include Spheromers CA6 ex. Microbeads AS, SunPPMA-COCO130, SunPPMA-COCO170, SunPPMA-P and SunPPMA-PH ex. Sunjin Chemicals, and the Celluflows C-25 and TA-25 ex. JNC Corporation.

Preferably, the dry shampoo comprises an oil which is preferably present at from 0.1 to 5% wt. of the base.

Preferably, the oil is isopropyl myristate, benzyl alcohol, PPG-3 benzyl ether myristate. The most preferred is isopropyl myristate.

Preferably, the dry shampoo comprises propylene glycol. Preferably any propylene glycol is present at from 0.5 to 5% wt. of the base.

In a composition according to the invention the propylene glycol reduces or prevents white residues being deposited on the hair. This is particularly problematic for users with dark hair.

The composition of the invention also comprises a propellant to suspend the base immediately before mixing and to facilitate its egress from its container.

The composition is stored in a pressurised container. Preferably, the container is an aerosol canister. More preferably, the container is an un-lacquered aerosol canister.

Suitable propellants are well known in the art and include butane, isobutene and propane.

### EXAMPLE 1

A virgin hair switch was treated with the control by way of applying sebum directly to the switch.

The switch was then treated with a dry shampoo composition comprising one of the test particles and the particles then removed with a magnet. The amount of sebum remaining was then measured as a percentage of that which was applied.

### EXAMPLE 2

Spheromers CA6 ex. Microbeads AS,

SunPPMA-COCO130, SunPPMA-COCO170, SunPPMA-P and SunPPMA-PH ex. Sunjin Chemicals,

Celluflows C-25 and TA-25 ex. JNC Corporation,

Styrene/Divinyl benzene

Styrene/Divinyl benzene and lauryl methacrylate (1:0.1)

Styrene/Divinyl benzene and 2-ethylhexyl metacrylate (1:0.1)

Styrene/Divinyl benzene and butyl acrylate (1:0.1)

Styrene/Divinyl benzene and lauryl methacrylate (1:0.3)

### EXAMPLE 3

Porous particles comprising styrene/divinyl benzene and optionally acrylate were prepared by suspension polymerization in a 1 L reactor equipped with a mechanical stirrer and nitrogen inlet according to the recipes shown in Table 1. The heterogeneous reaction mixture was degassed for half an hour whilst being stirred at 500 rpm. The nitrogen source was then raised above the solution and the stirring rate was dropped to 350 rpm. The reaction was allowed to proceed for 18 h at 70° C. The resulting porous particles were collected by filtration and washed by distilled water and ethanol. The particles were then left in 1 L of acetone solution stirring overnight to completely remove any linear polymer. The final product was filtered again and dried in vacuo for 24 hours at 60 ° C.

TABLE 1

| | Sample 1 | | 2 | | 3 | | 4 | | 5 | |
|---|---|---|---|---|---|---|---|---|---|---|
| Test particle | | | | | | | | | | |
| Divinyl- | 40 | g | 40 | g | 40 | g | 40 | g | 40 | g |
| benzene | | | | | | | | | | |
| (DVB) | | | | | | | | | | |
| Lauryl | | | 4 | g | | | | | | |
| methacrylate | | | | | | | | | | |
| 2-Ethylhexyl | | | | | 4 | g | | | | |
| methacrylate | | | | | | | | | | |
| Butyl | | | | | | | 4 | g | | |
| acrylate | | | | | | | | | | |
| Other | | | | | | | | | | |
| components | | | | | | | | | | |
| AIBN | 0.6 | g | 0.6 | g | 0.6 | g | 0.6 | g | 0.6 | g |
| Toluene | 100 | ml | 150 | ml | 150 | ml | 150 | ml | 200 | ml |
| Polyvinyl | 12 | g | 12 | g | 12 | g | 12 | g | 12 | g |
| alcohol | | | | | | | | | | |
| NaCl | 12 | g | 12 | g | 12 | g | 12 | g | 12 | g |

### EXAMPLE 4

The following are formulations for dry shampoos comprising particles. They are made by standard processes and comprise any of the particles described herein.

| | Dry Shampoo | Inventive Dry |
|---|---|---|
| Ingredient | Control | Shampoo |
| SD Alcohol 40-B (Alcohol Denat.) | 13.510 | 13.510 |
| Isopropyl Myristate | 0.250 | 0.250 |
| Aluminium starch octenyl | 1.875 | — |
| succinate | | |
| Test Particles | — | 1.875 |
| Silica | 0.065 | 0.065 |
| Fragrance (Parfum) | 0.300 | 0.300 |
| Isobutane (85%), Propane (15%) | 84 | 84 |
| | 100.000 | 100.000 |

### EXAMPLE 5

Demonstrating the efficacy of styrene/divinyl benzene and lauryl methacrylate (1:0.3) particles in sebum removal and volume generation.

### Sebum Removal

| | DS control | Inventive DS* |
|---|---|---|
| Before DS application | 100% | 100% |
| After DS application | 25% | 32% |

*containing styrene/divinyl benzene and lauryl methacrylate (1:0.3) particles instead of aluminium starch octenyl succinate.

The data shows that the styrene/divinyl benzene and lauryl methacrylate (1:0.3) particles are superior to starch for sebum removal.

### Volume

Volume is not just a style preference but also a measure for cleansing. The more volume returned the greater the cleansing efficacy.

| | Front Volume | Side Volume | Average | |
|---|---|---|---|---|
| | mm² | mm² | Volume mm² | Volume % |
| Control | 12187.144 | 10373.923 | 11280.5335 | 100 |
| Sebum | 3904.388 | 2428.708 | 3166.548 | 28.07091 |
| DS | 4954.91 | 3674.479 | 4314.6945 | 38.24903 |
| control | | | | |
| Inventive | 8496.415 | 7974.322 | 8235.3685 | 73.00513 |
| DS | | | | |

The data again shows that styrene/divinyl benzene and lauryl methacrylate (1:0.3) particles are superior to starch.

### EXAMPLE 6

Demonstrating the efficacy of SunPPMA-PH particles in sebum removal and volume generation.

### Sebum Removal

| | Comparative | |
|---|---|---|
| | DS | Inventive DS* |
| Before DS application | 100% | 100% |
| After DS application | 45% | 0% |

*containing SunPMMA PH particles instead of aluminium starch octenyl succinate.

### Volume

| | | | Average | |
|---|---|---|---|---|
| | Front Volume | Side Volume | Volume | |
| | mm² | mm² | mm² | Volume % |
| Control | 12187.144 | 10373.923 | 11280.5335 | 100 |
| Sebum | 3904.388 | 2428.708 | 3166.548 | 28.07091 |
| DS | 8641.725 | 4862.074 | 6751.8995 | 59.85443 |
| control | | | | |
| SunPMMA | 9793.021 | 9476.537 | 9634.779 | 85.41067 |
| PH | | | | |

### EXAMPLE 7

Demonstrating the efficacy of Celluflow C25 particles in sebum removal and volume generation.

### Sebum Removal

| | Comparative | |
|---|---|---|
| | DS | Inventive DS* |
| Before DS application | 100% | 100% |
| After DS application | 25% | 50% |

*containing Celluflow C25 particles instead of aluminium starch octenyl succinate.

### Volume

| | Front Volume | Side Volume | Average | |
|---|---|---|---|---|
| | mm² | mm² | Volume mm² | Volume % |
| Control | 12187.144 | 10373.923 | 11280.5335 | 100 |
| Sebum | 3904.388 | 2428.708 | 3166.548 | 28.07091 |
| DS | 4954.91 | 3674.479 | 4314.6945 | 38.24903 |
| control | | | | |
| Celluflow | 8808.389 | 6282.224 | 7545.3065 | 66.88785 |
| C-25 | | | | |

### EXAMPLE 8

Demonstrating the efficacy of *Celluflow TA25 particles in sebum removal and volume generation.

### Sebum Removal

| | Comparative | |
|---|---|---|
| | DS | Inventive DS* |
| Before DS application | 100% | 100% |
| After DS application | 25% | 28% |

### Volume

| | Front Volume | Side Volume | Average | |
|---|---|---|---|---|
| | mm² | mm² | Volume mm² | Volume % |
| Control | 12187.144 | 10373.923 | 11280.5335 | 100 |
| Sebum | 3904.388 | 2428.708 | 3166.548 | 28.07091 |
| DS | 4954.91 | 3674.479 | 4314.6945 | 38.24903 |
| control | | | | |
| Celluflow | 8377.119 | 7587.96 | 7982.5395 | 70.76385 |
| TA-25 | | | | |

1. Dry shampoo composition comprising a base and propellant, wherein the base comprises sebum absorber particles, said particles comprising acrylate cross-polymer, polydivinyl benzene polymers or hydrophobically modified polysaccharide derivatives, wherein the particles are insoluble in water.

2. Composition according to claim 1 wherein the base comprises from 5 to 20% wt. of the composition.

3. Composition according to any preceding claim wherein the base comprises from 2 to 75% wt base sebum absorber.

4. Composition according to any preceding claim wherein the sebum absorber comprises particles of average particle size from 20 to 120 micrometer.

5. Composition as claimed in any preceding claim, wherein the particles are crosslinked at a level of preferably 20-90%, more preferably 50-90, most preferably 80-90%.

6. Composition according to any preceding claim wherein the particles comprise styrene/polydivinylbenzene copolymer.

7. Composition according to any preceding claim wherein the acrylate is a hydrophobically modified (meth)acrylate.

8. Composition according to claim 7 wherein the (meth)acrylate is alkyl methacrylate wherein alkyl means having from 1 to 22 carbons.

9. Composition according to any of claims 1 to 5, wherein the particles are selected from the group consisting of divinylbenzene, lauryl methacrylate, 2-ethylhexyl methacrylate, butyl acrylate and mixtures thereof.

10. Composition according to any one of claims 1 to 5, wherein the hydrohobically modified polysaccharide derivatives are hydrophobically modified cellulose derivatives.

11. Composition according to claim 10, wherein the hydrophobically modified cellulose derivative is selected from cellulose triacetate and cellulose acetate butyrate.

12. Composition according to any preceding claim comprising from 0.1 to 5% wt. oil.

13. Composition according to claim 11 wherein the oil is isopropyl myristate.
